(19) United States
(12) Patent Application Publication
(10) Pub. No.: US 2022/0096432 A1
(43) Pub. Date: Mar. 31, 2022

(54) METHOD FOR TREATING ZIKA VIRUS INFECTION WITH QUERCETIN-CONTAINING COMPOSITIONS

(71) Applicant: Quercis Pharma A.G., Zug (CH)

(72) Inventor: Thomas Christian Lines, Oberwil-Lieli (CH)

(21) Appl. No.: 17/549,343

(22) Filed: Dec. 13, 2021

Related U.S. Application Data
(63) Continuation of application No. 16/287,049, filed on Feb. 27, 2019, now abandoned.
Application No. 16/287,049 is a continuation of application No. 15/420,695, filed on Jan. 31, 2017, now abandoned.
(60) Provisional application No. 62/290,741, filed on Feb. 3, 2016.

(51) Int. Cl. A61K 31/366; A61K 31/352; A61K 31/375; A61K 31/455; A61K 31/519; A61K 31/7048

(52) U.S. Cl. CPC A61K 31/366; A61K 31/352; A61K 31/375; Y02A 50/30; A61K 31/519; A61K 31/7048; A61K 31/455

(57) ABSTRACT

A method for treating Zika virus infection with a composition containing quercetin or isoquercetin, together with one or more of vitamin B3, vitamin C, and a folate compound is described. Methods of preventing microcephaly and treating and preventing infections of other Flavivridae viruses are also described.

### CROSS-REFERENCE TO RELATED APPLICATIONS

The present application is a continuation of U.S. patent application Ser. No. 16/287,049 filed on Feb. 27, 2019, which is a continuation of U.S. patent application Ser. No. 15/420,695 filed on Jan. 31, 2017, now abandoned, which claims the benefit of U.S. Provisional Patent Application Ser. No. 62/290,741 filed on Feb. 3, 2016. The contents of the prior applications are hereby incorporated by reference in their entireties.

### BACKGROUND

The Flaviviridae family of viruses is a diverse group of human and animal pathogens, many of which cannot be effectively treated with existing therapies. The family contains four genera, i.e., Flavivirus, Pestivirus, Pegivirus, and Hepacivirus.

Among the viruses in the Flavivirus genus are West Nile virus, dengue virus, tick-borne encephalitis virus, yellow fever virus, and the Zika virus.

Zika virus is transmitted primarily by Aedes aegypti mosquitoes. In addition, Zika virus has been shown to be transmitted by mingling of bodily fluids or blood during sexual contact between humans at least one of whom has been infected by the Zika virus. Other Flaviviridae family members may also be transmitted via this route.

Zika virus infection can cause acute onset of fever, maculopapular rash, arthralgia, and nonpurulent conjunctivitis. These symptoms usually last from several days to one week. In some patients, Zika virus infection leads to Guillain-Barré syndrome, an autoimmune disorder of the nervous system.

Importantly, maternal-fetal transmission of Zika virus has been documented throughout pregnancy. Zika virus infection in pregnant women has been associated with fetal loss and microcephaly. Currently, there is no known effective treatment for Zika virus infection.

The need exists to develop treatments for Flaviviridae family virus infections in general and a Zika virus infection in particular.

### SUMMARY

To meet the need set out above, a method is provided for treating Zika virus infection by administering to a subject in need thereof an effective amount of a quercetin-containing composition, which also includes one or more of vitamin B3, vitamin C, and a folate compound. Also disclosed is a method for treating Zika virus infection using a composition containing isoquercetin and one or more of vitamin B3, vitamin C, and a folate compound. Additionally, another aspect features a method for treating Zika virus infection using an anti-viral drug together with the above-described compositions.

The details of one or more embodiments are set forth in the description below. Other features, objects, and advantages will be apparent from the description and from the claims.

### DETAILED DESCRIPTION

Embodiments disclosed herein are directed to a method of treating a subject infected with a Flavivirus, the method comprising administering a quercetin composition of embodiments herein. In some embodiments, the method comprises administering a composition comprising quercetin and one or more of vitamin B3, vitamin C, and a folate compound. In some embodiments, the Flavivirus may be selected from West Nile virus, dengue virus, tick-borne encephalitis virus, yellow fever virus, and the Zika virus.

Embodiments disclosed herein are based, in part, on the unexpected findings that quercetin, together with one or more of vitamin B3, vitamin C, and a folate compound, exhibits synergistic inhibition of Zika virus intracellular protein production and infectious virus production in a Zika virus-infected subject. In some embodiments, the compositions of embodiments herein prevent cell reprogramming by the Zika virus, and may help prevent microcephaly and related neurological disorders.

Accordingly, in some embodiments, a method for treating a subject infected by Zika virus comprises administering to the subject an effective amount of a composition comprising quercetin in combination with one or more of vitamin B3, vitamin C, and a folate compound. In some embodiments, the method comprises administering a composition comprising quercetin and vitamin B3. In another embodiment, the method comprises administering a composition comprising quercetin and vitamin C. Further, Zika virus may be treated using a composition that includes quercetin, vitamin B3, and vitamin C. In an additional embodiment, the compositions of embodiments herein may also contain a folate compound, such as vitamin B9, folate, pteroylglutamic acid, L-methyl folate, or a combination thereof. Additionally, in some embodiments, luteolin, epigallocatechin gallate (EGCG), or both can be added to the compositions described in embodiments herein.

The compositions of embodiments herein may also be used to prevent or reduce the risk of infection by Zika virus. In some embodiments, a method of preventing and/or reducing the risk of infection by a Zika virus comprises administering to an subject at risk of contracting the Zika virus a composition comprising quercetin and one or more of vitamin B3, vitamin C, and a folate compound. In some embodiments, the method comprises administering a composition comprising quercetin, vitamin B3, and vitamin C. In some embodiments, the method comprises administering a composition comprising quercetin, vitamin B3, vitamin C, and a folate compound. In some embodiments, the subject at risk of contracting the Zika virus may be a healthy subject. In some embodiments, the subject at risk of contracting the Zika virus may be a healthy subject. In some embodiments, the subject at risk of contracting the Zika virus may be a pregnant subject. In some embodiments, the subject at risk of contracting the Zika virus may be a fetus.

In some embodiments, a method of treating a subject infected by a Zika virus comprises co-administering the compositions described herein with an anti-viral drug. It is believed that co-administration to a subject of the compositions with an anti-viral drug reduces the side effects associated with the anti-viral drugs (e.g., fever, fatigue/myalgias, headache, nausea, arthralgias, depression, skin rash, neutropenia, anemia, thrombocytopenia, and birth defects) and advantageously allows for a lower dose of these drugs to be used to treat Zika virus infection. The term “co-administration” refers to simultaneous administration or sequential administration of two different treatment modalities. The phrase “sequential administration” refers to administering a second composition soon after a first composition. For example, the second composition can be administered 30 minutes, 1 hour, 2 hours, or 4 hours after administration of the first composition. In some embodiments, the anti-viral drug may be administered before, after, or simultaneously with the quercetin compositions of embodiments herein. In some embodiments, the antiviral drug and the quercetin composition of embodiments herein may be administered as a single composition. In some embodiments, the quercetin composition of embodiments herein may further comprise an antiviral drug.

In some embodiments, the anti-viral drug is a protease inhibitor, an interferon, a nucleoside analogue, a polymerase inhibitor, an immune modulator, or a combination thereof. In some embodiments, the anti-viral drug administered is selected from abacavir, acyclovir, adefovir, amantadine, amprenavir, ampligen, arbidol, atazanavir, balavir, cidofovir, dolutegravir, darunavir, delavirdine, didanosine, docosanol, edoxudine, efavirenz, emtricitabine, enfuvirtide, entecavir, ecoliever, famciclovir, fomivirsen, fosamprenavir, foscarnet, fosfonet, ganciclovir, ibacitabine, imunovir, idoxuridine, imiquimod, indinavir, inosine, integrase inhibitor, interferon type III, interferon type II, interferon type I, interferon, lamivudine, lopinavir, loviride, maraviroc, moroxydine, methisazone, nelfinavir, nevirapine, nexavir, nitazoxanide, nucleoside analogues, novir, oseltamivir, peginterferon alfa-2a, penciclovir, peramivir, pleconaril, podophyllotoxin, protease inhibitor, raltegravir, reverse transcriptase inhibitor, ribavirin, rimantadine, ritonavir, pyramidine, saquinavir, sofosbuvir, stavudine, synergistic enhancer (antiretroviral), telaprevir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine, or a combination thereof.

Embodiments herein are also directed to the prevention, reducing the risk, and/or treatment of microcephaly in a subject infected with the Zika virus comprising administering to the subject a composition of embodiments herein. In some embodiments, a method of preventing microcephaly in a subject at risk thereof comprises administering to the subject a composition comprising a quercetin composition of embodiments herein. In some embodiments, the method comprises administering a composition comprising quercetin, vitamin B3, and vitamin C. In some embodiments, the method comprises administering a composition comprising quercetin, vitamin B3, vitamin C and a folate compound. In some embodiments, the subject at risk thereof is a fetus. In some embodiments, a method of preventing microcephaly in a fetus comprises administering to a pregnant woman carrying the fetus a composition of embodiments herein. In some embodiments, the compositions of embodiments herein may also be used to treat or prevent the birth defects associated with microcephaly. In some embodiments, the compositions of embodiments herein may also be used to treat or prevent the associated neurological disabilities of microcephaly. In some embodiments, the compositions disclosed herein may be used to treat or prevent impaired cognitive development, delayed motor functions and speech, facial distortions, dwarfism or short stature, hyperactivity, seizures, difficulties with coordination and balance, and other brain or neurological abnormalities associated with microcephaly.

In some embodiments, the compositions described herein may also be used to treat subjects infected with other viruses from the Flaviviridae family. Examples of these viruses include, but are not limited to, dengue fever virus, West Nile fever virus, yellow fever virus, St. Louis encephalitis virus, Japanese encephalitis virus, Murray Valley encephalitis virus, tick-borne encephalitis virus, Kunjin encephalitis virus, Rocio encephalitis virus, Russian spring summer encephalitis virus, Negeishi virus, Kyasanur forest virus, Omsk hemorrhagic fever virus, Powassan virus, Louping Ill virus, Rio bravo virus, Tyuleniy virus, Ntaya virus, Modoc virus, GB virus A, GB virus C, GB virus D, bovine viral diarrhea virus, swine fever virus, hog cholera virus, Hep C virus, and Hep G virus. Embodiments herein are directed to a method of treating a subject infected with a Flaviviridae virus comprises administering to the subject an effective amount of a composition of embodiments herein. For example, a method of treating a subject infected with a Flaviviridae virus comprises administering to the subject an effective amount of a composition comprising quercetin. In some embodiments, a method of treating a subject infected with a Flaviviridae virus comprises administering to the subject an effective amount of a composition comprising quercetin and one or more of vitamin C and vitamin B3. In some embodiments, a method of treating a subject infected with a Flaviviridae virus comprises administering to the subject an effective amount of a composition comprising quercetin, vitamin C and vitamin B3. In some embodiments, a method of treating a subject infected with a Flaviviridae virus comprises administering to the subject an effective amount of a composition comprising quercetin, vitamin C, vitamin B3, and a folate compound.

Treatment of subjects in need thereof with the compositions described above can lessen negative side effects caused by replication of the Flaviviridae virus. In another aspect, the compositions can prevent or reduce the risk of infection by any of these viruses.

The efficacy of quercetin is enhanced by vitamin B3, vitamin C, or both. For example, a combination of quercetin, vitamin B3, and vitamin C maintains quercetin levels in plasma up to five times those of quercetin alone or a combination of quercetin and vitamin B3. Further, a combination of quercetin, vitamin B3, and vitamin C results in a quercetin half-life in plasma twice as long as that of quercetin alone and about one and a half times that of a combination of quercetin and vitamin B3. See U.S. Pat. Nos. 7,745,486 and 7,745,487. A folate compound, such as L-methyl folate (also known as 5-methyltetrahydrofolate or METAFOLIN™), improves the efficacy of quercetin, as well as the efficacy of quercetin together with vitamin B3, vitamin C, or both.

In some embodiments, the composition may be administered in an amount that provides about 20 mg to about 5 g, about 100 mg to about 5 g, about 250 mg to about 5 g, about 20 mg to about 1 g, about 100 mg to about 1 g, about 200 mg to about 1 g, or about 250 mg to about 1 g of quercetin per day. In some embodiments, the composition may be administered in an amount that provides about 250 mg to about 5 g of quercetin per day. In some embodiments, the composition may be administered, once or periodically per day, in an amount that provides about 500 mg to about 5 g of quercetin per day. In some embodiments, the composition may be administered in an amount of about 20 mg, about 50 mg, about 100 mg, about 150 mg, about 200 mg, about 250 mg, about 250 mg, about 500 mg, about 750 mg, about 1 g, about 1.5 g, about 2 g, about 2.5 g, about 3 g, about 3.5 g, about 4 g, about 4.5 g, about 5 g, or a range between any two of these values. In some embodiments, the composition provides about 2 g of quercetin per day. In some embodiments, the composition provides about 5 g of quercetin per day.

When vitamin B3, vitamin C, or folic acid is included in the composition, each dose or serving may include about 20 μg to about 3 g vitamin B3, about 200 μg to about 3 g vitamin C, or about 40 to about 3000 μg of a folate compound. The amount of vitamin B3, vitamin C, and the folate compound in the composition may depend on the amounts of the other ingredients, i.e., quercetin, vitamin B3, folate compound, and vitamin C. More specifically, it depends on the intended amounts of all 4 ingredients per dose or serving.

In some embodiments, the composition comprises vitamin B3 in each dose or serving in an amount of about 10 μg to about 5 g, about 10 μg to about 3 g, about 15 μg to about 3 g, about 20 μg to about 3 g, about 100 μg to about 3 g, about 200 μg to about 3 g, about 300 μg to about 3 g, or a range between any two of these values. In some embodiments, the vitamin B3 in each dose or serving is about 20 μg to about 3 g.

In some embodiments, the composition comprises vitamin C in each dose or serving in an amount of about 100 μg to about 5 g, about 200 μg to about 5 g, about 300 μg to about 5 g, about 100 μg to about 3 g, about 150 μg to about 3 g, about 200 μg to about 3 g, about 300 μg to about 3 g, or a range between any two of these values. In some embodiments, the vitamin C in each dose or serving is about 200 μg to about 3 g.

In some embodiments, the composition comprises a folate compound in each dose or serving in an amount of about 40 to about 3000 μg, about 100 to about 3000 μg, about 40 to about 1500 μg, about 100 to about 1500 μg, about 40 to about 1200 μg, about 100 μg to about 1200 μg. In some embodiments, the composition comprises a folate compound in each dose or serving in an amount of about 40 μg, about 75 μg, about 100 μg, about 250 μg, about 500 μg, about 750 μg, about 1000 μg, about 1200 μg, about 1500 μg, about 2000 μg, about 2500 μg, about 3000 μg, or a range between any two of these values. In some embodiments, the folate compound in each dose or serving is about 100 μg, about 1200 μg.

In some embodiments, the weight ratio between quercetin, vitamin B3, and vitamin C in a composition of embodiments herein may be 1:0.02-1:0.2-2.5, or any ratio in between. For example, the weight ratio can be 1:0.04-0.5:0.3-2.0, 1:0.05-0.3:0.4-1.5, 1:0.05-0.2:0.5-1, and 1:0.1-0.2:0.5-1. In some embodiments, the weight ratio of quercetin, vitamin B3, and vitamin C may be 1:0.02:1, 1:0.04:1, 1:0.08:1, 1:0.16:1, or a range between any two of these values.

In some embodiments, the weight ratio between isoquercetin, vitamin B3, and vitamin C in a composition of embodiments herein may be 1:0.02-1:0.2-2.5, or any ratio in between. For example, the weight ratio can be 1:0.04-0.5:0.3-2.0, 1:0.05-0.3:0.4-1.5, 1:0.05-0.2:0.5-1, and 1:0.1-0.2:0.5-1. In some embodiments, the weight ratio of isoquercetin, vitamin B3, and vitamin C may be 1:0.02:1, 1:0.04:1, 1:0.08:1, 1:0.16:1, or a range between any two of these values.

In some embodiments, the composition may be administered once, twice, thrice, or periodically per day. In some embodiments, the composition may be administered about 2 to about 4 times per week. In some embodiments, the composition of embodiments herein may be administered in a single or in multiple doses. In some embodiments, the composition of embodiments herein may be administered over a 1, 2, 3, or 4 week period.

In some embodiments, the composition consists essentially of quercetin, vitamin B3, and vitamin C as the only active ingredients. In some embodiments, the composition consists essentially of isoquercetin, vitamin B3, and vitamin C as the only active ingredients.

In some embodiments, the composition consists essentially of quercetin, vitamin B3, vitamin C, and folic acid as the only active ingredients. In some embodiments, the composition consists essentially of isoquercetin, vitamin B3, vitamin C, and folic acid as the only active ingredients.

In some embodiments, the composition includes quercetin, vitamin B3, and vitamin C as the only active ingredients. In some embodiments, the composition includes isoquercetin, vitamin B3, and vitamin C as the only active ingredients.

In a particular embodiment, the composition includes quercetin, vitamin B3, vitamin C, and folic acid as the only active ingredients. In another embodiment, the composition includes isoquercetin, vitamin B3, vitamin C, and folic acid as the only active ingredients.

The term “quercetin” refers to quercetin aglycon and quercetin derivatives including, but not limited to, quercetin-3-O-glucoside (isoquercetin), quercetin-5-O-glucoside, quercetin-7-O-glucoside, quercetin-9-O-glucoside, quercetin-3-O-rutinoside, quercetin-3-O-[α-rhamnosyl-(1→2)-α-rhamnosyl-(1→6)]-β-glucoside, quercetin-3-O-galactoside, quercetin-7-O-galactoside, quercetin-3-O-rhamnoside, quercetin-3-O-β-D-glucopyranoside (isoquercitrin), and quercetin-7-O-galactoside. After digestion, quercetin derivatives are converted to quercetin aglycon and other active derivatives, which are absorbed in the body. These quercetin aglycones and other active derivatives can be subsequently sulfated, methylated, glucuronylated, and/or glucosidated, among other modifications.

The quantity of quercetin mentioned above refers to that of quercetin aglycon or the quercetin moiety of a quercetin derivative. Quercetin may be added to the composition either in a pure form or as an ingredient in a mixture (e.g., a plant extract). Examples of commercially available quercetin include QU995 (99.5% pure quercetin) and QU985 (98.5% pure quercetin) from Quercegen Pharmaceuticals LLC (Marlborough, Mass.).

Also commercially available from Quercegen Pharmaceuticals LLC are ISQ 950 AN (95% pure isoquercetin), ISQ 995 AN (99.5% pure all-natural isoquercetin) and ISQ 995 CIT (99.5% pure isoquercitrin).

“Vitamin B3” mentioned herein includes vitamin B3 in its various forms, including niacinamide, nicotinic acid, nicotinamide, and inositol hexaniacinate.

“Vitamin C” mentioned herein includes vitamin C (i.e., L-ascorbic acid, D-ascorbic acid, or both) and its salts (e.g., sodium ascorbate).

“Folate compound” mentioned herein includes pteroylglutamate and related compounds including tetrahydrofolate, vitamin B9, folate, pteroylglutamic acid, L-methyl folate (also known as 5-methyltetrahydrofolate or METAFOLIN™), salts thereof and derivatives thereof. As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The term “consisting essentially of” means the method or composition includes the steps or components specifically recited, and may also include those that do not materially affect the basic and novel characteristics of the present invention.

The compositions described above can be in various forms. For example, it can be a soft chew composition that includes quercetin, niacinamide, ascorbic acid, sodium ascorbate, folic acid, sugar, corn syrup, sucralose, soy lecithin, corn starch, glycerin, palm oil, xylitol, carrageenan, FD&C Yellow #6, FD&C Yellow #5, and natural and/or artificial flavors. An exemplary serving of this soft chew composition (5.15 g) includes 250 mg of quercetin, 12.9 mg of vitamin B3 (i.e., niacinamide), and 382.8 mg of vitamin C (i.e., L-ascorbic acid and sodium ascorbate). In some embodiments, a subject can take 1 to 20 servings (e.g., 4 servings) of this soft chew composition daily. The amounts taken may vary depending on, for example, the disorder or condition to be treated and the physical states of the subject. Another exemplary composition of this soft chew includes 5.25 wt % of quercetin, 0.25 wt % of vitamin B3, and 7.81 wt % of vitamin C (i.e., L-ascorbic acid and sodium ascorbate) plus 200 μg of folic acid per chew.

The composition may further contain one or more active ingredient, such as an isoflavone (e.g., genistein or genistin), curcumin, resveratrol, luteolin, epigallocatechin gallate (EGCG), coenzyme Q10, eicosapentaenoic acid (EPA), and docosahexaenoic acid (DHA). In some embodiments, a composition may include luteolin, EGCG, or both, in addition to quercetin, vitamin B3, vitamin C, and a folate compound. These active ingredients may be added to the composition either in a pure form or as a component in a mixture (e.g., an extract from a plant or an animal). A suitable daily dosage of each of these ingredients may vary depending on, for example, the disorder or condition to be treated and the physical states of the subjects. Exemplary daily dosages of some of these ingredients include: 20-2,500 mg (preferably 250-1,000 mg) of curcumin, 10-1,000 mg (preferably 100-500 mg) of resveratrol, 50-1,000 mg (preferably 100-700 mg) of EGCG, 25-300 mg (preferably 50-100 mg) of genistin/genistein, 10-1,000 mg (preferably 100-200 mg) of luteolin, 50-1,000 mg (preferably 70-500 mg) of EPA, and 50-1,000 mg (preferably 80-700 mg) of DHA.

When the composition of embodiments herein is in powder form, it may be used conveniently to prepare beverage, paste, jelly, capsules, pills, or tablets. Lactose and corn starch may be used as diluents for capsules and as carriers for tablets. Wetting agents, emulsifiers and lubricants, such as sodium lauryl sulfate and magnesium stearate, as well as coloring agents, release agents, coating agents, sweetening, flavoring and perfuming agents, preservatives and anti-oxidants can also be present in the compositions.

The compositions of embodiments herein may be prepared as a pharmaceutical composition or compositions in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The compositions set forth, supra, may be in the form of a dietary supplement or a pharmaceutical formulation. As a dietary supplement, additional nutrients, such as minerals or amino acids may be included. In some embodiments, a pharmaceutical formulation can be a sterile injectable or infusible solution that includes the composition together with pharmaceutically acceptable excipients. This solution may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol.

The composition may also be a food product. As used herein, the term “food” broadly refers to any kinds of liquid and solid/semi-solid materials that are used for nourishing humans and animals, for sustaining normal or accelerated growth, or for maintaining stamina or alertness. Examples of human food products include, but are not limited to, tea-based beverages, juice, coffee, milk, jelly, cookies, cereals, chocolates, snack bars, herbal extracts, dairy products (e.g., ice cream, and yogurt), soy bean product (e.g., tofu), and rice products.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

The terms “improving,” “enhancing,” “treating,” and “lowering” refer to the administration of an effective amount of the above-described compositions to a subject who needs to improve one or more of the above-mentioned conditions or has one or more of the just-mentioned disorders, or a symptom or a predisposition of one of more of the disorders or conditions, with the purpose to improve one or more of these conditions, or to prevent, cure, alleviate, relieve, remedy, or ameliorate one or more of these disorders, or the symptoms or the predispositions of one or more of them. The term “administration” covers oral or parenteral delivery to a subject of the above-described compositions in any suitable form, e.g., food product, beverage, tablet, capsule, suspension, and solution. The term “parenteral” refers to subcutaneous, intracutaneous, intravenous, intramuscular, intraarticular, intraarterial, intrasynovial, intrasternal, intrathecal, intralesional, and intracranial injection, as well as various infusion techniques. An “effective amount” refers to a dose of the composition that is sufficient to provide a therapeutic benefit (e.g., reducing the levels of Zika virus in the serum). Both in vivo and in vitro studies can be conducted to determine optimal administration routes and doses.

The compositions described above can be preliminarily screened for their efficacy in treating the above-described conditions by in vitro assays and then confirmed by animal experiments and clinic trials. Other suitable analytical and biological assays are apparent to those of ordinary skill in the art. For example, the effectiveness of the compositions described above can be measured by conducting in vitro viral replication studies.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method for treating Zika virus infection, the method comprising administering to a subject in need thereof an effective amount of a composition that includes quercetin and one or more of vitamin B3, vitamin C, and a folate compound.

2. The method of claim 1, wherein the composition includes vitamin B3.

3. The method of claim 2, wherein the composition includes vitamin C.

4. The method of claim 3, wherein quercetin, vitamin B3, and vitamin C are the only active ingredients administered to the subject.

5. The method of claim 2, wherein the composition includes a folate compound.

6. The method of claim 5, wherein quercetin, vitamin B3, vitamin C, and the folate compound are the only active ingredients administered to the subject.

7. The method of claim 1, wherein the composition includes vitamin C.

8. The method of claim 7, wherein the composition includes a folate compound.

9. The method of claim 2, wherein the composition includes a folate compound.

10. The method of claim 1, wherein the composition further includes one or more of genistein, genistin, curcumin, resveratrol, luteolin, epigallocatechin gallate (EGCG), coenzyme Q10, eicosapentaenoic acid (EPA), and docosahexaenoic acid (DHA).

11. The method of claim 10, wherein the composition includes luteolin or epigallocatechin gallate (EGCG).

12. A method of preventing or reducing the risk of microcephaly in a fetus, the method comprising administering to a pregnant woman carrying the fetus a composition comprising quercetin, and one or more of vitamin B3, vitamin C, and a folate compound.

13. The method of claim 12, wherein the composition includes vitamin B3.

14. The method of claim 13, wherein the composition includes vitamin C.

15. The method of claim 14, wherein quercetin, vitamin B3, and vitamin C are the only active ingredients administered to the subject.

16. The method of claim 14, wherein the composition includes a folate compound.

17. The method of claim 16, wherein quercetin, vitamin B3, vitamin C, and the folate compound are the only active ingredients administered to the subject.

18. The method of claim 12, wherein the composition includes vitamin C.

19. The method of claim 18, wherein the composition includes a folate compound.

20. The method of claim 13, wherein the composition includes a folate compound.

21. The method of claim 12, wherein the composition further includes one or more of genistein, genistin, curcumin, resveratrol, luteolin, epigallocatechin gallate (EGCG), coenzyme Q10, eicosapentaenoic acid (EPA), and docosahexaenoic acid (DHA).

22. The method of claim 21, wherein the composition includes luteolin or epigallocatechin gallate (EGCG).

23. The method of claim 1, wherein the quercetin is isoquercetin.

24. The method of claim 12, wherein the quercetin is isoquercetin.
